(19) United States
(12) Patent Application Publication
(10) Pub. No.: US 2024/0226447 A1
(43) Pub. Date: Jul. 11, 2024

(54) SYSTEM AND METHOD FOR DISTRIBUTING ROSIN AND OTHER VISCOUS MATERIALS

(71) Applicant: Konstantine P. Kralis, Skokie, IL (US)

(72) Inventor: Konstantine P. Kralis, Skokie, IL (US)

(21) Appl. No.: 18/406,704

(22) Filed: Jan. 8, 2024

Related U.S. Application Data
(60) Provisional application No. 63/437,964, filed on Jan. 9, 2023.

(51) Int. Cl. A61M 5/315; A61K 31/00; A61K 36/185; A61M 5/32

(52) U.S. Cl. CPC A61M 5/31548; A61K 31/658; A61K 36/185; A61M 5/3202; A61M 2205/6081

(57) ABSTRACT

A dosage unit for distributing viscous material comprises threaded cartridge and tip 6, cartridge casing 7, threaded top 8 with insert 9, threaded cap body 10, piston rod and indicator 11, and window 12 for indicating the amount of viscous material remaining in the dosage unit. The dosage unite modifies Novo Nordisk's “dosage unit” for use with viscous materials.

### CROSS REFERENCE TO RELATED APPLICATIONS

This application claims the benefit of Provisional Application No. 63/437,964 filed on Jan. 9, 2023, which is hereby incorporated by reference in its entirety.

### BACKGROUND OF THE INVENTION

This application relates to and describes an improved system and method for distributing rosin, plant-based oils, plant-extracted concentrates, and other viscous materials. Many of these viscous materials have temperature dependent viscosity levels that vary depending on temperature. The system and method can also be applied to water-soluble products. Use of extracted rosin and extracted plant-based oils has recently become popular in connection with marijuana/cannabis, but other plant-based materials can also be extracted as rosin or oil. For example, rosin can be obtained from the oleoresin or deadwood of pine trees or from tall oil, and the oils extracted from that process can be used for making varnish. Rosin can be a translucent, sappy material (sometimes referred to as “shatter”), an oil-like substance, a budder or batter (more like a solid), or a wax-like material. The consistency of the rosin depends on the starting material and the details of the particular process used to extract the rosin, among other factors. The present invention is applicable to any type of rosin or plant-based oil capable of being stored in the dosage unit described herein.

The devices and applications disclosed herein are described in the context of cannabis rosin and cannabis oil, although their applicability to rosin, plant-extracted concentrates, oils derived from other plants, and other viscous materials will be apparent to those of ordinary skill in the art.

Prior art mechanical dosage units exist for the injection and or distribution of liquids, such as insulin, in a precise format. For example, Novo Nordisk's “dosage unit” described in EP0327910 and WO1999/02210 provides an easy-to-use yet precise system and method for storing and properly dosing insulin to patients. Applicant provides herewith copies of EP0327910 and WO1999/02210 which are herein incorporated by reference, and those references should be considered part of this application, including as described herein with certain modifications that build upon the existing Novo Nordisk dosage unit. The Novo Nordisk dosage unit is designed for use with water-based materials but not oil-based materials. A need exists in the art for a similar dosage unit that can be used to store and distribute rosin, oil, plant extracts, or other viscous materials. In particular, consumers that ingest or inhale cannabis oils containing THC and/or CBD would prefer to have an easy and precise system for distributing desired amounts of the cannabis oils (and correspondingly, desired amounts of THC and/or CBD). This becomes especially important for patients that use THC or CBD oils for medical treatment, although it is also important for many recreational users of THC or CBD.

### SUMMARY

The system and method described herein provides a mechanical dosage unit for storing and distributing rosin, oil, plant extracts, or other viscous materials, and in particular, materials with temperature-dependent viscosities. The mechanical dosage unit described herein is also suitable for storing and distributing water-based materials such as mushrooms, medications, or ayahuasca brews.

The figures, block diagrams, and other attachments and explanations provided herewith include additional technical details of the dosage unit.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts.

The use of the words “function,” “means” or “step” in the Detailed Description or Description of the Drawings or claims is not intended to somehow indicate a desire to invoke the special provisions of 35 U.S.C. § 112, ¶6, to define the invention. To the contrary, if the provisions of 35 U.S.C. § 112, ¶6 are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. § 112, ¶6.

### BRIEF DESCRIPTION OF THE SEVERAL VIEWS OF THE DRAWINGS

A more complete understanding of the present invention may be derived by referring to the detailed description when considered in connection with the following illustrative figures. In the figures, like reference numbers refer to like elements or acts throughout the figures.

FIG. 1 depicts an isometric view of a pen without the cap body 10 and the top 8.

FIG. 2 depicts an isometric view of a pen without the cap body 10 and the top 8, including a piston rod 11 outside of the pen.

FIG. 3 depicts an isometric view of a pen with the cap body 10 and the top 8 disassembled, and with the cartridge and tip 6 removed.

FIG. 4 depicts an isometric view of a pen without the cap body 10 and the top 8, including a piston rod 11 outside of the pen.

FIG. 5 depicts an isometric view of a pen with the cap body 10 sliding onto the pen and the top 8 disassembled, including a piston rod 11 outside of the pen.

FIG. 6 depicts an isometric view of the cartridge and tip 6.

FIG. 7 depicts a partial isometric view of the cartridge casing 7 and the cartridge and tip 6.

FIG. 8 depicts an isometric view of top 8 and insert 9.

Elements and acts in the figures are illustrated for simplicity and have not necessarily been rendered according to any particular sequence or embodiment.

Embodiments depicted are shown only schematically, and not all features may be shown in full detail or in proper proportion. Certain features or structures may be exaggerated relative to others for clarity. The embodiments shown are examples only, and should not be construed as limiting the scope of the present disclosure or appended claims.

### DETAILED DESCRIPTION

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, devices and technologies to which the disclosed inventions may be applied. The full scope of the inventions is not limited to the examples that are described below.

The existing Novo Nordisk dosage unit (described in expired patents EP0327910 and WO1999/02210, which are incorporated by reference herein) has several flaws that make it unsuitable for use with cannabis oils, other oils, or other viscous materials. The present disclosure describes several key improvements upon that existing Novo Nordisk dosage unit to adapt it for use with cannabis oils and other oils.

One problem with the Novo Nordisk dosage unit is that when higher viscosity materials (and in particular materials with temperature dependent viscosities), such as cannabis oils, are stored in the cartridge, the piston rod of the dosage unit is unable to properly function to push liquid out of the cartridge. For example, when the button is pushed to displace the piston rod, the needle tip, other portions of the tip, or cartridge may be pushed out of the dosage unit (and may break) rather than pushing out the desired dose of liquid from the tip. In short, the viscosity or “hardness” of the oil at room temperature may be too high for the Novo Nordisk dosage unit, without modification, to function properly.

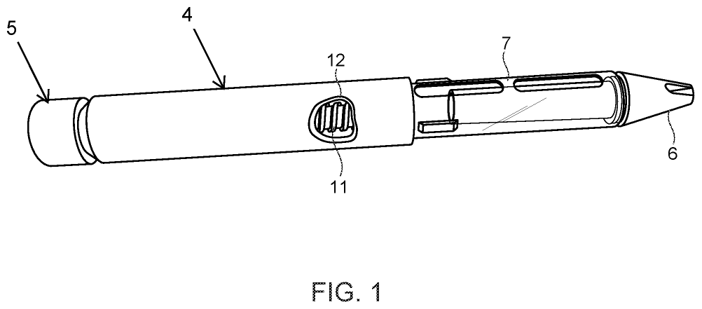

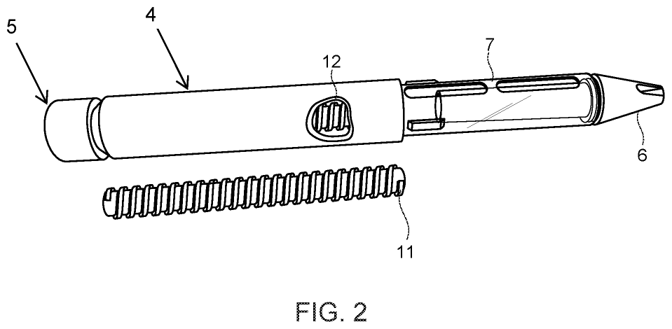

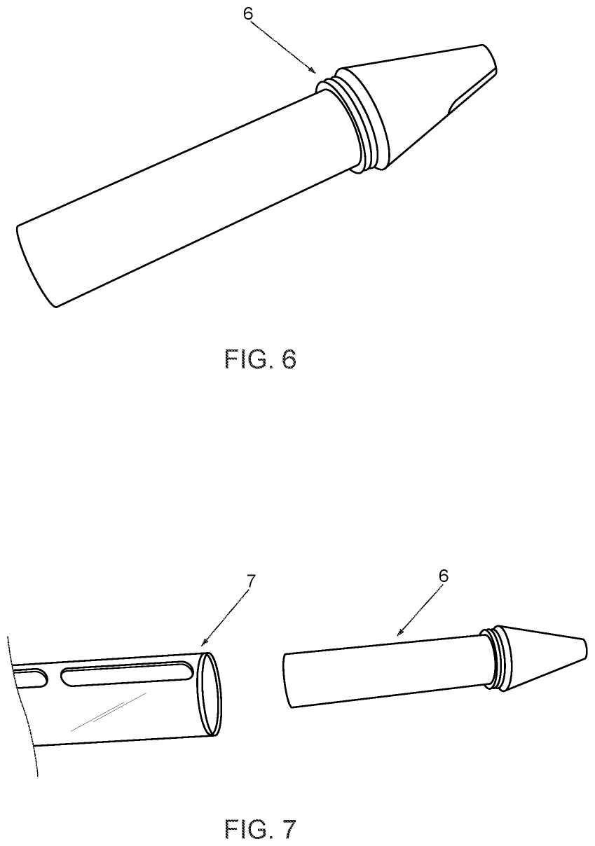

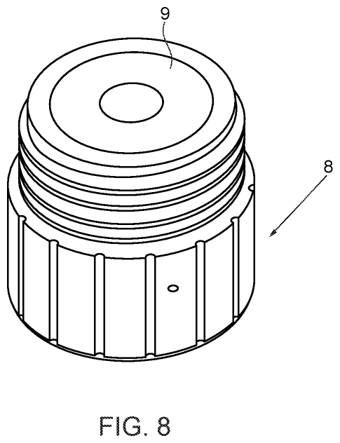

To solve this problem, one aspect of the dosage unit disclosed herein and shown in FIGS. 1, 2, 4, 6, and 7 is an improved tip and cartridge. Rather than snapping a needle into a cartridge and then snapping the cartridge into the dosage unit, this aspect of the dosage unit includes a one-piece tip and cartridge 6. The tip and cartridge 6 includes threads (in the provided example, male threads near its tip). The cartridge casing 7 (depicted in FIGS. 1-5) includes corresponding threads (in the example, female threads) so that the tip and cartridge 6 is screwed into the cartridge casing 7. This provides a more safe and secure cartridge and tip while still using the existing piston rod and corresponding system for controlling the piston rod to push oil out of the cartridge and tip 6. In other words, the existing prior art piston rod and plunger for the piston rod fit into the improved cartridge and tip 6, but the improved cartridge and tip 6 is more securely fastened to the cartridge casing 7 to allow the piston rod to properly push oil out of the cartridge and tip 6.

In the example shown in the figures, the cartridge and tip 6 is machined from aluminum, although other known materials are also suitable.

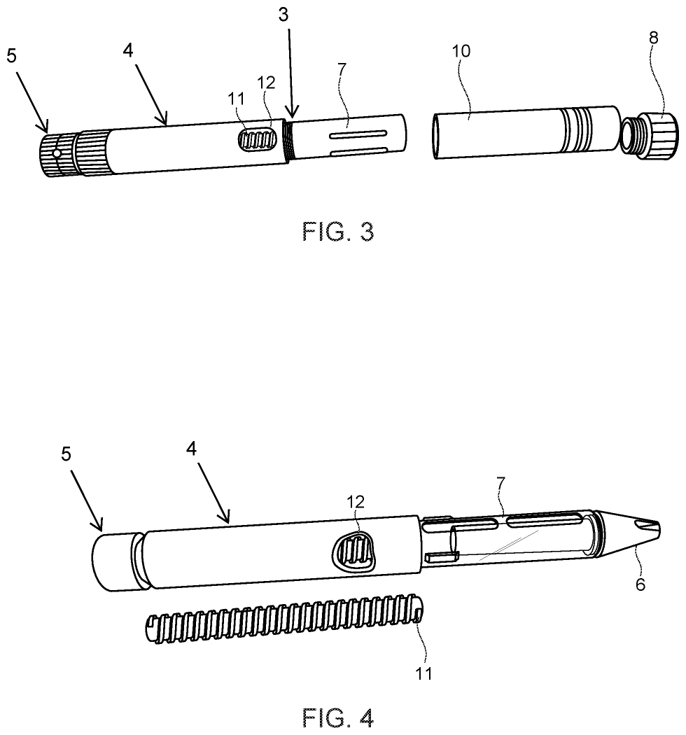

Another problem with the Novo Nordisk dosage unit is that oils stored in the cartridge undesirably leak from the dosage unit tip. Another aspect of the improved dosage unit described herein provides for an improved cap that prevents such leaking. The cap (depicted in FIGS. 3, 5, and 8) has an adjustable, threaded top 8 that includes an insert 9 that will be in contact with the cartridge and tip 6. The insert 9 is typically made from softer materials such as silicone or foam. In particular, a foam insert 9 may be more desirable for cold climates, and a silicone insert 9 may be more desirable for warm climates. Other materials are also suitable. One user of the dosage unit might have multiple threaded tops 8 (for example, each with a different insert 9 material) that can be switched out depending on the particular application or climate.

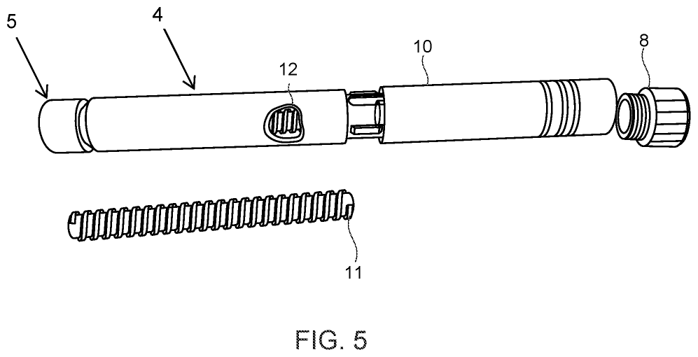

The threaded top 8 screws into a threaded cap body 10 (depicted in FIGS. 3 and 5). Because of the threads, the top 8 can be screwed into the cap body 10 until the insert 9 is in contact with the tip of the cartridge and tip 6. The pressure can be increased by continuing to screw the top 8 into the cap body 10, or decreased as desired. This allows provides the capability to adjust the pressure of the insert 9 on the cartridge and tip 6 to prevent oils from leaking out of the cartridge and tip 6.

The improved cap body also includes all of the prior art features of the Novo Nordisk dosage unit cap that it needs to pair with and turn the “adjustment means” (reference numeral 18 in EP0327910, incorporated by reference herein) so that desired dose can be set and pushed out of the dosage unit.

Specifically, EP0327910 describes at 6:25-7:26 describes how the adjustment means 18 in EP0327910 is used to cause the indicator 21 in EP0327910 to protrude from the back of the dosage unit, showing an approximate dosage amount on the end of the dosage unit near the end button 22. The user then removes the cap 24 in EP0327910. As described in EP0327910, “Then the indicator 21 is forced back into the adjustment means 18 by pressing on the end button 22 until said movement is stopped by the abutment of the nut means 13 against the end of the casing 1 or the abutment of the end button 22 against the adjacent end of the adjustment means. As the pawl 11 prevents the piston rod 8 from rotating, the displacement of the indicator 21 causes a displacement of the piston rod a corresponding distance, whereby the piston of the cartridge is pressed toward the outlet end of the liquid 3. As a result, a quantity of liquid is pressed out of the cartridge, said quantity corresponding to the quantity measured on the measuring scales.”

The dosage unit described herein includes the same mechanism for causing a dose to be pushed out of the cartridge and tip 6, with the body 4 shown in FIGS. 1-5 corresponding to the adjustment means 18 in EP0327910, the button 5 shown in FIGS. 1-5 corresponding to the end button 22 in EP0327910, and the piston rod 11 shown in FIGS. 1-5 corresponding to the piston rod 8 in EP0327910. The internal components of EP0327910 described in the quoted passage above are also used in the present invention, although they are not shown for simplicity.

A problem with the system in EP0327910 is that the cap 24 is not tightly screwed on to the pen. As a result, in a pen system that uses oil or other viscous materials, such as the system described herein, a high risk exists for leaking or losing the pen cap 24.

As depicted in FIG. 3, one aspect of the dosage unit described herein is a threaded end 3 of the body 4 that can mate with a threaded end of the cap body 10 so that the cap screws tightly on to the dosage unit. This prevents leakage. In particular, adjustable threaded top 8 and threaded body 4 can be used together so that the cap is both tightly connected to the body 4 and tightly pressed against the cartridge and tip 6 to prevent oils from leaking out of the cartridge and tip 6.

With this arrangement, the cap body 10 must be removed in order to adjust the body 4 so that the indicator can protrude from the back of the dosage unit and the button 5 can be used to push out a dose. However, with the cap off of the dosage unit, the cartridge casing 7 can be rotated relative to the body 4 to cause the indicator to protrude from the back of the dosage unit in the same fashion as described above, simply without the need for the cap. In other words, by rotating the casing 7 relative to the cap body 4, dosing works in the same way as described in EP0327910. The difference is that the threads 3 of body 4 provide a much tighter seal for the dosage unit when the dosage unit is not in use, especially when combined with adjustable threaded top 8 with insert 9.

Another aspect of the dosage unit described herein is a color-coded piston rod 11 and window 12 in the dosage unit. In this aspect (depicted in FIGS. 1-5), the piston rod 11 is color-coded, but it is otherwise the same as the piston rod used by the Novo Nordisk dosage unit. As doses are released from the dosage unit (i.e., pushed out by the piston rod with a plunger in the cartridge and tip 6), different colors of the piston rod 11 show through the window 12 because the piston rod 11 is pushed toward the cartridge and tip 6 each time a dose gets distributed. As an example for how the piston rod 11 can be color coded, the first part of the piston rod 11 can be green, so that the green portion of the piston rod 11 shows through the window 12 before the dosage unit is used to distribute any oil, and the green portion continues to show until the first 25% of the doses have been distributed. The center part of the piston rod 11 can be a neutral color, such as white or yellow. In this example, the neutral color will be shown in the window 12 after 25% of the doses have been distributed and until 75% of the doses have been distributed. The last part of the piston rod 11 can be red. The red will show in the window while the last 25% of the doses have been distributed. The exact colors used for the color coding and proportions of the color coding can be adjusted as desired. The color-coded piston rod 11 and window 12 provide a simple indication to the user of approximately how many doses remain in the cartridge.

As an alternative to color coding, numbers can be written on the piston rod 11 at various intervals to show a percentage or number of “clicks” remaining through the window 12.

The material in each cartridge has a specific flavor profile, potency (of THC and/or CBD, for example), viscosity (which can be temperature-dependent), plant profile, etc., and the user chooses the desired profile for a specific occasion or scenario.

Many applications exist for the precise dosing distribution system described herein in the form of a dosage unit, including direct oral dosing (medical or recreational) into a user's mouth (for example, sublingually), dosing product for vapor inhalation of heated drops (commonly referred to as “dabbing”), adding drops to edible foods or drinks (in a commercial or recreational setting), adding drops to cannabis or other products that are smoked (such as a joint or a glass receptacle filled with cannabis), or combining the product with other products in tinctures.

During dosing, the tip of the dosage unit can be held directly over a user's mouth, so that the dose lands on a user's tongue (or sublingually under a user's tongue) and is ingested directly. In the alternative, in cooking or baking processes that call for THC, CBD, plant-extracted concentrate, other rosin/oils, or other viscous materials, the dosage unit can be held over the pan, the mixing bowl, the food product, etc. and the material is distributed from the dosage unit to the food product. The dosage unit can be used for “dosing” drinks as well. This is a simple and accurate way to distribute a desired amount of rosin, plant-extracted concentrate, or oil (and correspondingly, a desired amount of THC or CBD)—for example.

In the foregoing Detailed Description, various features can be grouped together in several example embodiments for the purpose of streamlining the disclosure. This method of disclosure is not to be interpreted as reflecting an intention that any embodiment requires more features than are expressly recited in the corresponding claim. Rather, inventive subject matter may lie in less than all features of a single disclosed example embodiment. Thus, the present disclosure shall also be construed as implicitly disclosing any embodiment having any suitable set of one or more disclosed or claimed features (i.e., a set of features that are neither incompatible nor mutually exclusive) that appear in the present disclosure, including those sets that may not be explicitly disclosed herein.

The scope of the originally filed claims does not necessarily encompass the whole of the subject matter disclosed herein. The originally filed claims are hereby incorporated into the Detailed Description, with each claim standing on its own as a separate disclosed embodiment. The scope of subject matter encompassed by each claim shall be determined by the recitation of only that claim.

The conjunction “or” is to be construed inclusively (e.g., “a dog or a cat” would be interpreted as “a dog, or a cat, or both”; e.g., “a dog, a cat, or a mouse” would be interpreted as “a dog, or a cat, or a mouse, or any two, or all three”), unless: (i) it is explicitly stated otherwise, e.g., by use of “either . . . or,” “only one of,” or similar language; or (ii) two or more of the listed alternatives are mutually exclusive within the particular context, in which case “or” would encompass only those combinations involving non-mutually-exclusive alternatives. The words “comprising,” “including,” “having,” and variants thereof, wherever they appear, shall be construed as open ended terminology, with the same meaning as if the phrase “at least” were appended after each instance thereof.

If any one or more disclosures are incorporated herein by reference and such incorporated disclosures conflict in part or whole with, or differ in scope from, the present disclosure, then to the extent of conflict, broader disclosure, or broader definition of terms, the present disclosure controls. If such incorporated disclosures conflict in part or whole with one another, then to the extent of conflict, the later-dated disclosure controls.

The Abstract is provided as required as an aid to those searching for specific subject matter within the patent literature. However, the Abstract is not intended to imply that any elements, features, or limitations recited therein are necessary.

What is claimed is:

1. A dosage unit, comprising:
a. a cartridge containing a viscous plant material;
b. a piston configured to push a dose of the viscous plant material out of the cartridge by linear displacement of the piston toward an open tip of the cartridge;
c. a window in the dosage unit configured to show at least a portion of the piston, wherein the piston is color-coded to indicate in the window the amount of viscous plant material remaining in the dosage unit.

2. The dosage unit of claim 1, wherein the cartridge is threaded and configured to mate with a threaded portion of the body of the dosage unit.

3. The dosage unit of claim 1, wherein the dose is user-specified.

4. The dosage unit of claim 1, wherein the dose is specified by a user using a clicking mechanism of the dosage unit.

5. The dosage unit of claim 1, further comprising a removeable cap configured to cover the open tip of the cartridge.

6. The dosage unit of claim 5, wherein the removeable cap includes an insert that is configured to press against the open tip of the cartridge to close the open tip of the cartridge.

7. The dosage unit of claim 6, wherein the removeable cap includes a threaded portion usable for adjusting an amount of pressure exerted by the insert against the open tip of the cartridge.

8. The dosage unit of claim 5, wherein the removeable cap is configured to mate with the dosage unit via a threaded coupling.

9. The dosage unit of claim 1, wherein the viscous plant material is cannabis extract.

10. The dosage unit of claim 1, wherein a green color in the window indicates 75% or more of the viscous plant material remains in the dosage unit.

11. The dosage unit of claim 1, wherein a red color in the window indicates 25% or less of the viscous plant material remains in the dosage unit.

12. A dosage unit, comprising:
a. a cartridge containing a viscous plant material;
b. a piston configured to push a dose of the viscous plant material out of the cartridge by linear displacement of the piston toward an open tip of the cartridge;
c. a window in the dosage unit configured to show at least a portion of the piston, wherein the piston includes numbers to indicate in the window the amount of viscous plant material remaining in the dosage unit.

13. The dosage unit of claim 12, wherein the cartridge is threaded and configured to mate with a threaded portion of the body of the dosage unit.

14. The dosage unit of claim 12, wherein the dose is user-specified.

15. The dosage unit of claim 12, wherein the dose is specified by a user using a clicking mechanism of the dosage unit.

16. The dosage unit of claim 12, further comprising a removeable cap configured to cover the open tip of the cartridge.

17. The dosage unit of claim 16, wherein the removeable cap includes an insert that is configured to press against the open tip of the cartridge to close the open tip of the cartridge.

18. The dosage unit of claim 17, wherein the removeable cap includes a threaded portion usable for adjusting an amount of pressure exerted by the insert against the open tip of the cartridge.

19. The dosage unit of claim 16, wherein the removeable cap is configured to mate with the dosage unit via a threaded coupling.

20. The dosage unit of claim 12, wherein the viscous plant material is cannabis extract.
